(19) United States
(12) Patent Application Publication
(10) Pub. No.: US 2014/0214047 A1
(43) Pub. Date: Jul. 31, 2014

(54) Device and Method for Medical Records Storage in the Eye

(71) Applicants: Murad A. Sunalp, Tulare, CA (US); Donn K. Harms, Del Mar, CA (US)

(72) Inventors: Murad A. Sunalp, Tulare, CA (US); Donn K. Harms, Del Mar, CA (US)

(21) Appl. No.: 13/750,913

(22) Filed: Jan. 25, 2013

(51) Int. Cl. A61F 9/007; A61B 19/00

(52) U.S. Cl. CPC A61F 9/007; A61B 19/54
USPC 606/117

(57) ABSTRACT

A method and apparatus for the storage and retrieval of patient personal and medical information and records in the eye of an implanted or patient. Patient medical information or other personalized data in the form of indicia is imparted to an implant which is engaged into a mounted position in the eye during eye surgery or a needle insertion. The indicia may be read through the clear tissue of the eye, using an optical magnifier once implanted. Indicia is imparted to the implant using a laser or photographically or printing. Conventionally employed laser surgery equipment used for LASIK and similar surgeries can be easily adapted to both impart the indicia to the implant and form a cavity to hold the implant for subsequent viewing of the visible indicia.

This application is a Continuation-in-Part application from U.S. patent application Ser. No. 11/546,751 filed on Oct. 11, 2006, and included herein in its entirety by this reference thereto.

### FIELD OF INVENTION

The device and method herein provides for local storage and viewing of patient medical records and other patient information. More particularly, the disclosed device and method provide for the local storage and review of patient information such as medical records, on the patient's person, and readable without the need for a network connection or electronic data storage and retrieval. The device and method thereby provides vital and important information, about the patient or person, which is immediately legible without electronic or other means for communication.

The system is particularly well suited for data creation and implantation in a position rendering the data readable during emergencies, during a simple eye surgery such as when undergoing laser corrective eye surgery, or lens replacement surgery or a surgery employing a laser which may concurrently write the data to be made readable. Further the device and method are particularly well suited for use as a manner for identifying a patient using a quick review of their eye during the initial surgical team review during surgery, to prevent patient and surgical mistakes.

Employing the implanted and locally readable device, a method is provided wherein the device combined with portable computing device such as a pad computer or smartphone are employable as an interface and patient identifier for emergency medical professionals to view basic and other patient records and information locally, without the need for electronic network communication or radio communication to retrieve such information which is frequently lacking or made ineffective due to the inability to identify the patient. Still further, employing the device and method herein, supplemental patient records and information can be ascertained, even if the patient is unable to speak or communicate, to allow the responding or local medical professional to link to a service provider and retrieve more detailed information about the patient than what is provided locally. This is achieved with no patient input whatsoever since such is provided by the visible, readable implant, with no patient input needed.

### BACKGROUND

With the advent of computer-guided laser eye surgery and with the evolution of lens replacement surgery to a common occurrence an ever larger number of patients are undergoing such procedures to correct their vision. There are many other types of surgeries in addition to eye surgeries for improving vision, which employ lasers locally. However, procedures employing a laser such as for facial surgery, cosmetic surgery or to reshape the cornea have become a particular favorite with both patients and medical professionals. In the case of eye surgery, in such procedures an excimer or other laser is employed to reshape the eye's surface (cornea) to alter the way light rays enter the eye to achieve a correct focus. Laser-Assisted In Situ Keratomileusis (LASIK) treats nearsightedness, farsightedness, and astigmatism.

During a LASIK procedure the surgeon cuts a flap in the stroma, then employs an excimer laser to remove material under the flap. LASIK surgery usually takes less than one minute per eye.

Laser Epithelial Keratomileusis (LASEK) is similar to LASIK, except that the surgeon cuts a flap in the epithelium only, instead of through the epithelium and part of the stroma. The surgeon then loosens the edges of the flap with alcohol. LASEK is used mostly for people with thin or flat corneas who are poor candidates for LASIK, which requires more corneal tissue for success. Epi-LASIK is like LASEK surgery in that the flap is cut only in the epithelium, but the surgeon uses neither a blade nor alcohol. Instead, the surgeon uses an epikeratome, which is a separator that creates an epithelial sheet. Because no alcohol is involved, patients are likely to feel less pain than in alcohol procedures and will generally heal faster. Bladeless or “All Laser” LASEK is like LASIK, except that an additional laser instead of a bladed instrument (microkeratome) is used to create the flap in the front of the eye. As with regular LASIK, the flap then is lifted and laser energy is used to reshape the eye for vision correction.

With the many thousands of new patients for laser assisted surgeries such as facial or plastic surgery or laser eye surgery have also come thousands of medical professionals to provide such surgeries. As with any business, professionals in the medical field may change locations or may change affiliations or cease practice. Because many of the patients who undergo laser assisted surgeries may at some point in the future need a follow-up procedure, it is important that the records of any previous procedure be available to the subsequent operating medical professionals or facility.

However, with medical professionals retiring, changing practices or affiliations, moving or ceasing business, it can be extremely hard for a patient to retrieve their records from a prior laser or other eye procedure. Since medicine is an art and the medical professional makes medical decisions based on all available information, it is very important that any prior laser eye procedure records be available for review in determining a course of action in a subsequent procedure.

Because of the above-noted changes in practice and other reasons for lost or unavailable medical records, a problem may be present for the subsequent eye surgeon if they cannot view the records of any prior procedures. It is especially important to know how much of the eye surface might have been removed, the size of the flaps cut in the cornea on a prior occasion, and the prior vision history before a prior correction. Without this information, the subsequent surgeon may not be able to provide the best course of action for the patient on the subsequent surgery.

In addition, in recent years there has been an ongoing progression for digital medical records for patients which has been pushed by both government and medical providers. However, while the advent of digital records may be more convenient and accurate in a medical provider's office or a hospital, in emergency situations, there are numerous reasons digital medical records are not always the best choice.

First, should the patient be unconscious, or otherwise unable to identify themselves, first responders, emergency room personnel, and hospital or other medical personnel have no way to ascertain the patient's identity other than viewing their wallet or cellphone in the hopes of finding such. Even if an unknown patient is identified, such does not provide any immediate information concerning medical conditions requiring emergency or immediate attention, nor information about the location of a larger scope of patient information. Consequently, even if an unresponsive patient is identified, which takes valuable time during what is known as the “golden hour” where actions by first responders and emergency medical personnel can make the difference between life and death and the quality of life for the patient.

If first responders and medical personnel can at some point identify the patient, it can take precious hours to ascertain medical and personal information, that is required to treat the patient properly. Even responsive patients, in an emergency situation, or when injured, will be unable to provide their medical information with accuracy or in a fashion which will allow responders and medical treatment facilities to use good medical judgment and provide the best possible mode of emergency and subsequent treatment for that patient.

Still further, while some patients may carry some medical information on paper or electronic media on their person, if they are unresponsive it will not be easily found. Even if the patient is responsive, most electronic media requires the proper interface and electronic computing device to gain access to the digitized information. However, there is not easy device and method provided, which allows first responders as well as medical providers, to ascertain the actual presence of patient information on their person, which does not require patient input, or a search of the patient, or a disrobing and search of the patient, and inspection, to ascertain if any information is present, if such can be seen or found. Even if ascertained as present, without proper software and processors or other manner of reviewing the information, it may be unusable. Finally, many minutes or hours may go buy while the patent and their belongings are searched for such information and to translate it or discern it leaving the patient untreated or less than optimally treated for the entire time.

In addition, it is known that there currently exists problems with the proper identification of the patient during normal surgeries and medical procedures. For instance, surgery on an eye which is to be subject to the ophthalmic surgical procedure. Often, a patient is firstly diagnosed to determine the type and intensity of ophthalmic surgery needed for one or both eyes, and then scheduled for surgery at a later time. Currently, eye identification for the correct eye to undergo the procedure is provided by engaging an identifying marker, such as an adhesive sticker, onto a users forehead above the eye intended for surgery. Such identification and surgical procedure marking is employed with other surgical procedures. Such a method, which provides a low-tech solution, is far from fool proof. Mistaken identification, or loss of the identifying marker may occur. It is also known to happen that the patient may remove the marker due to skin irritation of the sticker adhesive, and/or replace it in the wrong position.

Unfortunately, with the current state of medical record availability and patient and surgical site identification, the information gathered from a diagnostic team may not be successfully communicated to the surgical team up to the time of operation. In some instances, a surgeon has been known to mistakenly operate on the wrong limb or body position or eye due to the surgeons and surgical staffs inability to identify the patient properly. In the case of eye surgery where one or both eyes are to be operated on, the surgeon may mistakenly perform an operation upon one eye which should have been performed on the other, and vis versa. This can result in even worse vision for the patient than prior to treatment, and the need for additional surgery.

In addition to the employment of an implant in the viewable portion of the patient's eye which is discernable by third parties, the system can also employ imaging of the eye or eyes of the patient themselves as a secondary means for identification or confirmation of discerned identification. In addition to inserting the implant into the cornea or sclera where it may be easily discerned as present, the service or professional will take digital photos of exterior and/or interior surfaces of the eye or eyes of the patient and capture biometric ophthalmic data. Much like a human finger print, the retina and the iris and portions viewable in the eye such as the macula or fovea and combinations thereof, are sufficiently unique in humans and animals to provide means for individual identification. A digital photo of the eye structure can be digitized and stored and later used to identify the individual when a subsequent digital photo is taken and compared to those stored in the database using software adapted to compare discerned markers for a match. Thus, in addition to providing a discernible, readable implant, the system can provide a secondary means to verify the identity using such captured and compared biometric ophthalmic data. Such makes available to users the ability to obtain patient information without patient input and without searching or otherwise disrobing the patient.

Using such captured biometric ophthalmic data, patient's undergoing surgery can be ascertained and the proper party by employment of a hand held computing device such as a smartphone, which would take digital photo of the eye or eye surfaces of the patient and then compare such to the stored digitized captured biometric ophthalmic data in the database for a cross match. This method would be especially good for use where eye surgery is to be performed, to ascertain if the correct eye is marked on the patient. However it could also be employed to ascertain the identity of non responsive patients, or patients undergoing regular surgery during a surgical team time out at the begriming of the operation where the eye can be photographed and transmitted to the surgical server which would communicate the procedure scheduled to the smartphone or other computer for comparison with what the surgical team believes is to be done.

Further, in addition to identification of patients for needed medical procedures for humans, and to provide a device which may be discerned by third parties as present, with no input from the patient or search thereof for possible information, the device and method herein described and disclosed can also be employed for storage and viewable retrieval of information and veterinary data for pets, dogs, cats, zoo animals, or expensive agricultural animals like bulls for breeding. Animals at present are unable to talk or otherwise reasonably communicate their identity, history, ownership, or any other information about themselves. While implantable chips or swallowed devices exist to store animal history and data, their presence cannot be easily discerned with a simple flashlight and instead they require an expensive electronic scanner to discern first if they are present, and second for the identification of the chip itself. Finally, the chip identity must be cross referenced in a database to patient information from a data library which requires network or other electronic communications, which frequently in emergencies such as tornados or earthquakes, are unavailable.

A device having a presence on a patient that may be visually discerned from a distance or a close proximity to the eyes of a patient with a simple light in both humans and animals, would be a significant advance over the current art of information carried in clothing and wallets and purses. An easily read device, requiring only conventional instruments for looking into the eye to read and retrieve important patient information immediately, would be of great benefit for humans and animals to provide an easy manner to ascertain their identity, and the important portions of their medical and other history. Such information is an important asset to medical professionals and to animal medical professionals and animal owners. Consequently, the herein disclosed method and apparatus for implantation of an easily discerned device and easily read device for records, which can be located and read without proprietary expensive scanners and network communications and the like and which can directly provide medical and other data regarding the recipient, would be of great value and immediately widely employable.

As such, there exists an unmet need for a method and apparatus that will allow for third parties to easily visually discern from a distance or close proximity to a patient, the existence of viewable discernible patient information on the patient's person. Such a device once visually discerned as existing, should be easily readable to discern important patient personal and medical information with conventional optical and medical equipment by direct viewing of the eye. Such a device should be capable of carrying the most important portions of the patient's medical, personal and surgical and treatment records for easy viewing, without the need for electronic or network communications, database lookups, and third party input.

Such a system should provide a manner to make the patient themselves the “cloud” or warehouse for their own important medical records of personal and medical information and prior and present conditions and procedures. Such a device and method should be employable with humans and animals and allow for easy visual unassisted identification for being present in each.

Still further, such a system and method should provide for easy ascertaining of supplemental patient information by a viewable identifiers for either the identity of the patient or of patient records, to allow for a network communication and lookup and provision of such supplemental records on a computer device communicating over a network. Both the ability to read and discern important patient and medical information, and the information to obtain supplemental records should be available without the need for expensive electronic scanning equipment by a simple viewing of written records and indica stored in a visually discernable location.

Still further, such a device and method should be ascertainable for its presence on the patient without a search of or any communication with the patient using the naked eye of a third party. For instance, such may be made discernable for presence from a distance or close proximity using one or a combination of a light illumination surface on the device which is visible to the naked eye by a third party such as one or a combination of a glowing, a fluorescence, at least one light reflective surface, fluorescing material, a prismatic shape to reflect refracted light, a radio frequency identification RFID, or the like. Therefor a viewing of the device in a patients eye can provide a means for positive identification of the patient, their medical conditions, and other important information in order to allow for effective treatment and mitigate surgical mistakes.

With respect to the above, before explaining at least one preferred embodiment of the invention in detail, it is to be understood that the invention is not limited in its application to the details of construction and to the arrangement of the components or steps set forth in the following description or illustrated in the drawings. The various apparatus and methods of the invention are capable of other embodiments and of being practiced and carried out in various ways which will be obvious to those skilled in the art once they review this disclosure. Also, the phraseology and terminology employed herein are for the purpose of description and should not be regarded as limiting.

Also, those skilled in the art will appreciate that the conception upon which this disclosure is based may be utilized as a basis for designing of other devices, methods and systems for carrying out the several purposes of the disclosed device. It is important, therefore, that the advantages and claims be regarded as including such equivalent construction and methodology in that they do not depart from the spirit and scope of this invention.

The forgoing examples of related art and limitation related therewith are intended to be illustrative and not exclusive, and they do not imply any limitations on the invention described and claimed herein. Various limitations of the related art will become apparent to those skilled in the art upon a reading and understanding of the specification below and the accompanying drawings.

### SUMMARY OF THE INVENTION

There is disclosed and described herein, a method and apparatus to store a patient's personal and/or medical information regarding eye surgery or other personal or medical records within the eye of the patient. The device and method feature a small inert plastic or synthetic implant on which indica regarding or identifying records pertinent to the patient's surgical, medical, or personal history may be directly imparted by a laser or by photographic, microprinting or other means. The implant would be sized for implantation into a small cavity that is formed in the cornea of the eye itself out of the viewing area of the eye. The implants can be employed in humans and in animals and in a particularly preferred embodiment would reveal their position in the eye without the aid of a magnifying means to allow the viewer to employ widely available eye viewing equipment to view the information thereon as it pertains to the person or animal in which it is implanted.

When employed to track a patient's eye surgery history, the inscription of patient data, or indicia identifying a repository for the data, can be imparted to the device by the same laser that is used for the eye surgery itself. Additionally, a cavity formed in the eye, for storage of the implant out of the viewing area of the cornea, can also be formed by the same laser during the surgery. Consequently, when used to store patient eye or other records and implanted during a laser eye surgery session, existing equipment already being used for such surgeries can be employed with software changes to impart the patient's history, or viewable history identification directly on the implant. Further, other information or identification my be imparted as desired or required.

The device and method can also be employed to hold data or record identification indica for animals or other types of human patients. When employed for data storage, the implant may have indicia written directly on the implant that may be viewed by simply using conventional eye viewing devices right through the cornea. The actual records themselves, or indicia indicating an accessible location for the records, can be read using such eye viewing instrumentation.

In a particularly preferred mode of the device and method, a material that luminesces under certain light wavelengths can be employed. This will allow the presence of the implant to be ascertained by simply shining the correct wavelength of light at the eye of the implantee, which will reveal the location of the implant by glowing. The device can then be viewed by appropriate magnification optical equipment for its information. Further, the device may be formed with one or a combination of a glowing or fluorescence material or surface and a light reflective surface such that presence of the implant can be viewed with the naked eye.

Subsequent to implantation and ascertaining its presence in the clear tissue of the cornea or eye and out of the field of view of the eye to avoid any impairment of vision, the implant may be easily “read” by any subsequent medical professional by simply viewing it through the surface of the eye with appropriate medical equipment like a microscope. This allows for subsequent medical professionals to ascertain the presence easily and to easily read a patient's prior history and surgery information, or ascertain a remote location using conventional equipment already available in most eye professional offices.

The indicia may be actual miniature reproductions of the indica and patient surgery chart or can be indica identifying a remote location to obtain the information. Either type of information should be easily “read” by viewing the implant through the clear eye tissue with the appropriate magnifying instrument. Such indicia can either be charts and drawings engraved on the implant itself, or numbers, letter, barcodes, or other means to provide an identification of a patient history record that may be obtained remote to the patient. One, two, and particularly three dimensional bar codes are particularly well suited to carry reproducible information since such bar codes may be encoded with data or images that are directly producible using appropriate software. Letters or numbers or one or two dimensional bar codes are well suited to identify a patient file history by an alphanumeric or other relational code, and a location identification in the form of a network address or phone number where the information relating to the identified implant, may be retrieved.

Further, in an additional preferred mode, a viewing of the device may provide a means for positive identification of a patient's eye prior to a ophthalmic surgical procedure, in order to mitigate surgical mistakes. This may be provided by a ‘reading’ of the device in a patient's eye which will provide a positive or negative identification of the eye as to whether or not to proceed with a surgical procedure. For example, a handheld electronic device, such as a smart phone, employing suitable software adapted at the task, can be employed to scan or capture an image of the user's eye, determine presence of the implant, as well as record or indicate patient information for retrieval by a surgical team at a later date. A secondary means for positive identification of the patient's eye may also be employed as a backup, such as an iris or retina scan providing a means for a ‘digital fingerprint’ of the eye for identification.

In accordance with at least one other preferred mode, a business method for retrieving patient information for emergency medical care is provided. In this method the implantation device can employ indicia of patient records in order to allow an emergency medical technician (EMT) to retrieve pertinent patient history without the need of a database in order to administer emergency care. The indicia may also include means to link to a service provider and download more detailed patient information for further care, such as via correct patient ID numbers and correct identification codes provided by the indicia for the inquirer being a medical facility or other professional.

In all embodiments of the device and method, ascertaining the presence of the device itself should be easily accomplished by looking for it implanted in the cornea or other clear tissue of the eye. Looking for it may be done by using conventional eye viewing medical equipment or illuminating the eye with the proper wavelength of light and looking for a glow. Also, in all embodiments of the device, either the information itself or means for identifying a file and location of the information is directly ascertainable by viewing a surface of the implant, through the clear tissue of the eye.

In this respect, before explaining at least one embodiment of the invention in detail it is to be understood that the invention is not limited in its application to the details of construction and to the arrangement of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other methods and systems for carrying out the several purposes of the present invention. It is important, therefore, that the claims be regarded as including such equivalent construction insofar as they do not depart from the spirit and scope of the present invention.

As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

It is an object of this invention to provide a method and apparatus for creating and mounting an implant for storage of a patient's medical records which can be placed directly into a cavity in a patient's eye, which is viewable from outside the eye.

It is a further object of this invention to provide such a method and apparatus using conventional laser and optical equipment already employed in most eye surgical centers.

It is a further object of this invention to provide such a medical and record keeping apparatus that may be read at a later date using magnification equipment that views the implant surface through the surface of the eye.

Yet an additional object of this invention is providing storage of other data and information regarding humans and animals in an outer surface of the eye viewable through that surface.

An additional object of this invention is the provision of such a record storing implant that luminesces under certain light wavelengths to reveal its presence to the naked eye.

A further object of this invention is the provision of an implant that is readable through the clear surface of the eye to reveal indicia identifying the specific implant in combination with a remote database of information pertaining to the implant identification indica.

Yet another object of the invention is the provision of a means for positive identification of a patient's eye prior to a ophthalmic surgical procedure.

A still further object of the invention is to provide a method for medical record retrieval for on site emergency medical care and means to provide detailed medical record retrieval for further or follow up medical care at a medical facility or the like.

There has thus been outlined, rather broadly, the more important features of the invention in order that the detailed description thereof may be better understood and in order that the present contribution to the art may be better appreciated. There are additional features of the invention that will be described hereinafter and which will form the subject matter of the claims appended hereto.

These together with other objects and advantages which will become subsequently apparent reside in the details of the construction and method as more fully hereinafter described and claimed, reference being had to the accompanying drawings forming a part thereof, wherein like numerals refer to like parts throughout.

### BRIEF DESCRIPTION OF THE DRAWING FIGURES

FIG. 1 depicts a conventional laser surgery wherein a flap is cut in the clear cornea and a laser employed to reshape the refractive surface of the cornea under the flap.

FIG. 2 depicts a laser imparting patient medical data, or implant identification information, to a small implant adapted to fit in a cavity or space outside the refractive surface of the cornea.

FIG. 3 shows the laser forming the cavity under the exterior surface of the eye and out of the refractive area or field of view of the eye.

FIG. 4 shows a perspective view of the implant mounted in the eye just below the exterior surface where its surface may be viewed by magnification or other equipment.

FIG. 5 depicts a typical medical chart showing the patient's prior eye surgical history and showing the size and location of any prior flaps in the eye, other medical information or data pertaining to the patient could be imparted.

FIG. 6 depicts a two-dimensional bar code which may be encoded with patient data and text which may be reproduced with proper decoding equipment once read through clear eye tissue.

FIG. 7 depicts indicia imparted to the viewable surface of the implant in the form of a bar code and alphanumeric characters.

FIG. 8 depicts an RFID engaged to the implant and alphanumeric characters imparted on the viewable surface of the implant.

FIG. 9 depicts a luminescent material engaged to the implant.

FIG. 10 depicts a flow diagram or a preferred method of the invention for retrieving patient information for on site emergency medical care, and retrieve detailed patient history for further care at a medical facility.

FIG. 11 shows yet another particularly preferred mode of the invention showing the device employed as a means for positive eye identification prior to surgery.

FIG. 12 shows a diagram of a preferred communication network of the device employed as a means for positive eye identification.

FIG. 13 depicts a mode of the implant formed of a material in a shape for prismatic reflections or of material which fluoresces or is luminescent under certain light spectrums directed at it providing a lighted visual cue of presence which works well in daylight but especially well at night to visually report its presence.

FIG. 14 shows a mode of an implant with readable indicia thereon, which may be formed of material which luminesces or fluoresces or with a reflective element which reflects colored or sparkling light to visually report the presence of the implant.

FIG. 15 depicts a magnifying lens attachment and illumination components which may be mounted to work with in conjunction with the camera lens of a smartphone or portable computer or camera, and optional fiber optic lens for extension of the magnifying lens.

FIG. 16 shows a mode of the device which includes a smartphone cover or portable computer case or cover, which has a magnification lens and illumination component positioned to register configured to work with the OEM camera lens to magnify the written indicia and display it on the display screen. Illumination components for strobe, UV, IR light or other noted light projection are also provided.

### DETAILED DESCRIPTION OF PREFERRED EMBODIMENTS

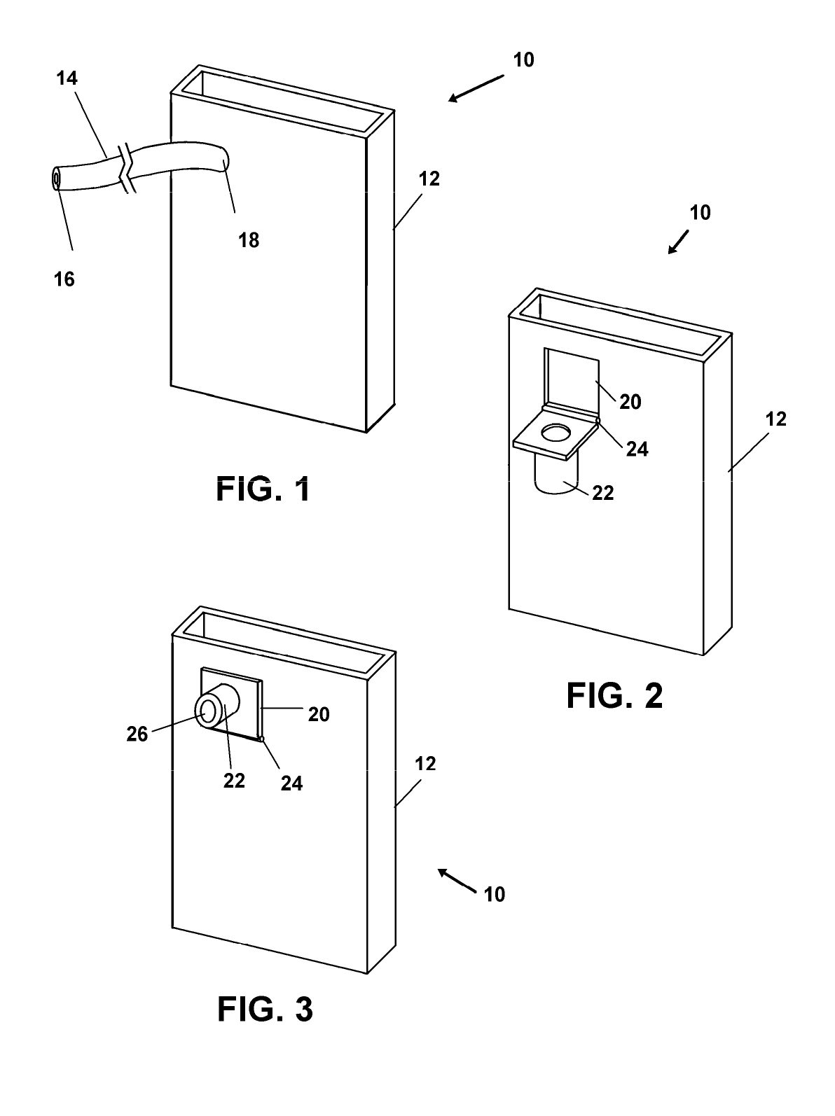

Referring now to the drawings in FIGS. 1-15, wherein similar parts are identified by like reference numerals, there is seen in FIG. 1 a perspective view of the eye during conventional laser surgery. During such a procedure, a flap 15 is cut in the cornea 17 or clear portion of the eye. The flap 15 is folded back to reveal a refractive portion of the cornea 17 in front of the lens and iris of the eye through which light is transmitted to the interior of the eye. A laser 14 is conventionally employed to reshape the refractive surface of the cornea under the flap 15 during such surgery.

Once the refractive area of the cornea 17 is reshaped, conventionally the flap 15 is replaced and heals.

In FIG. 2 there is shown one means to impart viewable indicia 19 to a front surface of the implant 12. As shown, the indicia 19 is imparted by writing it with a laser 14 on the viewable surface of the implant 12. The laser 12 may inscribe the indicia 19 into the surface itself if the implant 12 is formed of plastic or other material adapted to that purpose or onto a prepared surface coating adapted for laser inscription formed on the viewable surface. The current preferred mode of imparting indicia to the viewable surface of the implant 12 would be using the laser already employed at most medical and eye surgery centers. However, other means to impart the viewable indicia 19 such as printing, photography, or other means as would occur to those skilled in the art is anticipated. The key requirement is that the indicia 19 imparted to the viewable surface of the implant 12 is discernable looking through the clear eye tissue behind which it is mounted. Viewing would be by microscope or other optical equipment suitable to the task of viewing very small indicia on a very small surface through the clear tissue of the eye.

The indicia 19, which employed to inscribe or otherwise imparted upon the implant 12, in a particularly preferred mode would be the patient's medical data concerning the procedure being done. However, other medical data or information or images may be imparted to the viewable surface and such is anticipated since the implant may be employed as earlier noted for animals or humans for identification, tracking, or other purposes.

The indicia 19 so placed would be readable through the cornea or clear surface of the eye and would be alphanumeric in nature, or images or both. One, two, and three dimensional bar codes may also be employed. The indicia 19 may also be relational information which identifies the implant 12 using means to impart viewable indicia of individual identification which identifies a file of information related to the implant 12. Also included in the indicia 19 may be a remote location or file server which may be contacted to retrieve the information related to the individual identified implant 12. This will allow not only viewable information about the human or animal in which the implant 12 is placed, but also means to identify a remote file of information about the implant based on viewable indicia 19 seen by the viewer through the clear tissue of the eye. Unlike medical implants requiring a scanner and radio or other receiver to identify the presence of and identity of an implant, implantation of the disclosed device 10 may be ascertained by simply looking through the clear tissue of the eye.

A particularly preferred mode of the device as noted earlier, would have at least a portion 11 of the implant 12 formed of material adapted for photo luminescence when contacted by certain light spectrums such as ultra violet light. By simply shining the appropriate light spectrum in the eye, a viewing person looking through the cornea or clear tissue 17 of the implanted's eye may ascertain the presence of the device 10 without the need for magnification. Alerting to its presence without magnification will alert the professional to take further action and viewing. This component of the implant 12 would be especially helpful to any medical or other professional alerting them to the presence of a patient identifier and medical or other records that are accessible by viewing the information on the viewing surface 21. When implanted into animals or old or forgetful patients, anyone shining the appropriate light spectrum into the eye to luminesce the implant 12 will be immediately informed of its presence. Veterinarians can identify the animal without the need for special wands or receivers and can thereafter read the information directly from the implant 12 to ascertain the animal's identity and/or medical history. Doctors may do the same for patients which would be especially helpful of the patient is unable to communicate.

In the current preferred mode of the device 10, when implanted in eye surgery patients during laser surgery, indicia regarding the patient record data such as FIG. 5, or indicia identifying the patient and/or a remote location of patient record data and/or access codes or passwords as in FIGS. 6-8 is, in the current preferred mode, is placed upon the viewable surface 21 of the implant 12 by the laser 14 being used for the surgery itself. This can be accomplished with a simple software upgrade of existing laser eye surgery systems with some type of mounting to hold the implant 12 during inscription or impartation of the indicia 19 regarding one or a combination of patient records, remote record depository, patient identification, passwords to access remotely stored data, and/or other information, onto the viewable surface of the implant 12.

The indicia may be inscribed on the viewable surface 21 of the exterior surface of the implant 12 or within the interior of the implant 12. If the indicia is imparted to the interior body of the implant 12 by laser inscription, the viewable surface would need to be transparent so that the imparted indicia 19 would be viewable by a person through viewable surface 21 and through the clear tissue of the cornea 17 in which it is implanted. Interior impartation of indicia, may be preferable to avoid later aberrations in viewing the indicia and patient data should healing eye tissue cover the exterior of the implant 12. However, since such tissue is substantially clear like the surrounding tissue, viewing through it to the interior imparted data would work well.

As noted, other means for imparting indicia 19 to the viewable surface 21 of the implant 12 are anticipated so long as such means places indicia on the implant 12 which is viewable through the clear tissue of the eye using conventional optical means for magnification once the device 10 is implanted. Laser or ink printing, photographic, lithographic, or other means of imparting the indicia 19 that would occur to those skilled in the art are anticipated in the scope of this patent.

In a slightly less preferred mode of the device, the implant 12 could house an RFID 23 or other passive electronic memory holding device which does not require electric power to hold electronic memory. RFID's are particularly attractive as they may be energized to transmit their stored records, using exterior power, and hold a lot of data. While the preferred mode of the device uses a laser, photographic, or some other means to impart readable indicia on or into the implant 12, so it can be viewed in a low-tech manner by a simple microscope or other magnification means by any medical professional subsequently viewing into the eye tissue, RFID devices, while requiring software and communications abilities to receive the data stored, could also provide a means to store large amounts of medical or other data and information in the patient's eye for later retrieval thereby preventing complications from lost records and such.

If an RFID device is used to store electronic data, the implant 12 should provide indicia 19 on the viewable surface 21 through the clear tissue or cornea 17 of the eye, that provides a key or password to allow such a communication with the RFID somewhat like as in FIG. 8. The indicia 19 may also provide some means of identification of radio frequency or other required information needed to communicate with the secured RFID. This will allow the person reading the indicia 19 to ascertain the required communications protocols and passwords to receive and transmit information to the RFID. Positioning the identification information and/or other communication information on the viewable surface 21 thereby prevents unauthorized communication with the RFID and allows medical professionals or others to gain access by reading the access information through the clear tissue of the eye.

However, since imparting indicia 19 is easily achieved using the same laser 14 already employed to operate upon the patient, or microprinting, or other conventional means of imprinting, and since that indicia 19 would be easily read using conventional magnification means such as microscopes that are readily available in doctors' offices in industrialized nations as well as third world countries, laser or other means for directly imparting of the indicia 19 to the viewable surface 21 for carrying patient record data, or photographic or imprinting means of imparting the indicia 19, may be preferred as more usable in a low-tech environment and for the ability to retrieve the written information in a low-tech subsequent retrieval.

Once the human or animal patient data or other information desired for storage and easy viewing through the eye is imparted to the implant 12, or before, depending on the preference of the laser system manufacturer or whether the information will be electronically stored in addition to indicia 19, in a preferred mode of the device used during surgery, a cavity 16 is formed under the exterior cornea 17 or other clear surface area of the eye and out of the refractive field of view of the patient. The cavity 16 can be formed by the same laser 14 employed for the LASIK or similar surgery during reshaping of the cornea. The cavity 16 should as noted be formed out of the refractive area and in a current preferred mode would be located adjacent to the hinged area 13 of engagement of the flap 15. Locating the implant 12 in or adjacent to the hinged area 13 it has been determined, provides the most strength and least interference with subsequent eye surgeries if the flap 15 must be dismounted and is therefor a most preferred location; however, it is anticipated the implant 12 can be placed anywhere outside the refractive area of the cornea 17 for an operative installation such that the viewing surface 21 and or the luminescent area 11 may be seen from outside the eye. Currently, laser equipment employed for eye surgeries is tested by focusing the laser on a test piece of plastic or other test material and uses the focused beam for test cutting. The laser also is employed to cut or shape the cornea 17 according to calculated parameters for eyesight desired. Consequently, the installed base of laser eye surgery equipment in use already can be employed with software changes to direct the laser 14 to cut eye tissue to form the properly dimensioned and positioned cavity 16 to house the implant 12. Further, placing the implant in some type of holder and directing the laser to engrave or otherwise impart the indicia 19 readable from the viewable surface 21 is also achievable using current equipment with software modifications. If desirable, a separate laser might be employed adapted to the specific purpose of forming the cavity 16 during surgery and while the flap 15 is folded back, or to write the indicia 19.

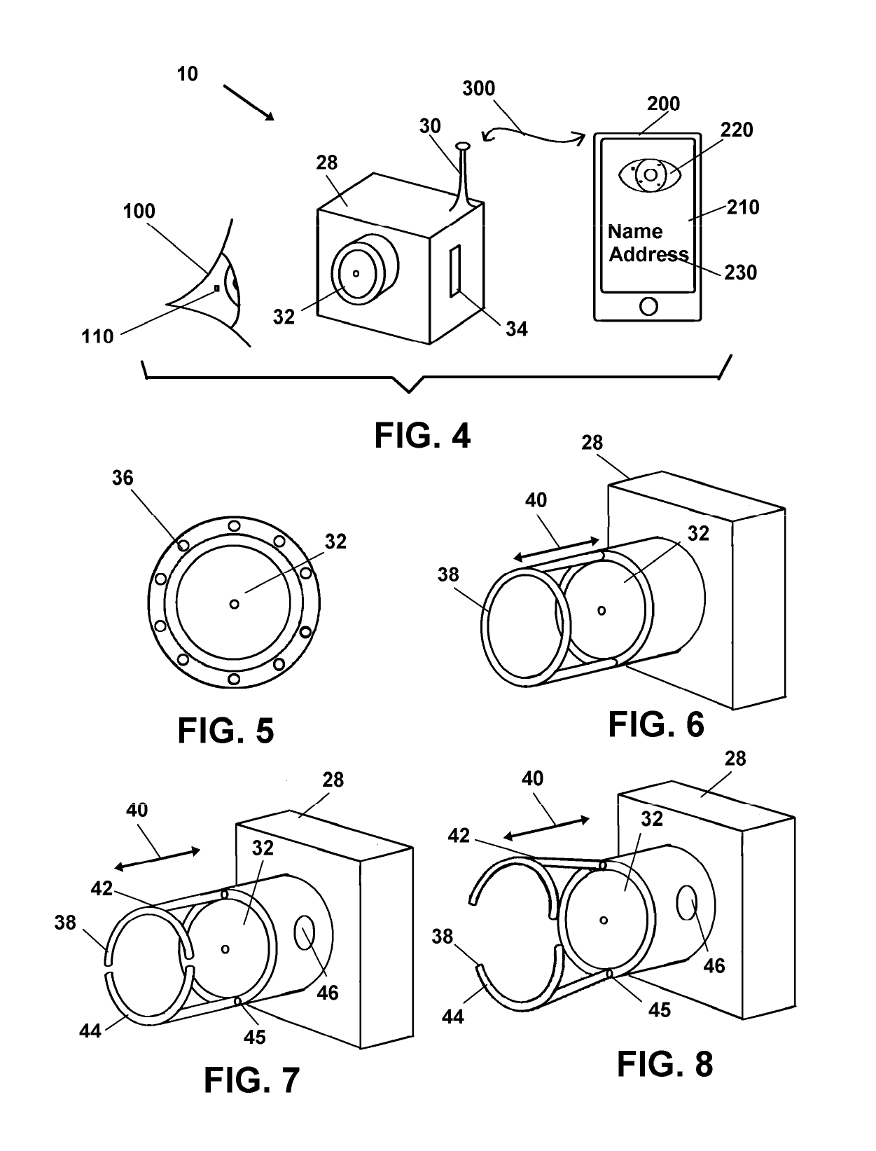

As depicted in FIG. 4 the implant 12 is mounted by the surgeon in the eye, in the formed cavity 16, just below the clear tissue on the exterior eye surface, where it may be viewed easily and directly using widely available magnification equipment. Subsequent medical professionals could as such simply look into the patient's eye and read the medical records imparted to the implant 12. They could also print it out using a printer adapted for engagement to the magnification equipment. Typical actual patient data imparted to the implant 12 is shown in FIG. 5 showing the patient's eye surgical history, and showing the size and location of any prior flaps 15 in the eye would be most helpful to subsequent medical professionals. As noted, this information would be easily “read” by subsequent medical professionals using conventionally available magnification equipment employed to examine eyes. As also indiciated elsewhere in the specification, other indicia 19 relating directly to the implanted, or a patient identity, and/or regarding remote locations of implanted data, and/or access codes to access implanted data stored remotely, as shown in FIGS. 6-8 for example, can also be placed on the implant 12 in a manner allowing viewing from outside the eye through the cornea 17 or clear eye tissue subsequent to implantation.

For employment in the eye when laser surgery is not being performed, the device 10 can be deployed in a microsphere injection into the stroma or cornea 17 or other clear tissue outside the refractive zone or by insertion into the clear area in other manners during a lens replacement surgery or other eye surgery or even during an eye or medical exam. Injectional placement in the eye would allow the implant 12 with readable indicia 19 on a viewable surface 21 to be placed in the eye at any time.

Subsequent to injection, with the proper viewing equipment noted above, the indicia 19 may be directly read through the clear tissue of the cornea 17 and information ascertained as to the individual implant 12 and/or implanted and/or location of remote information related to the implant 12 identified viewing the indicia 19 on the viewable surface through the clear eye tissue. Information as to access codes to access the information related to the identified patient and/or implant should also be visible to the person reading the viewable surface 21 of the implant through the cornea or clear tissue 17 of the eye. The injectable embodiment my have a broader scope of potential use in humans and animals as a means of identifying and tracking the implanted which may be initiated by viewing actual indicia 19 on the implant itself subsequent to implantation.

In a method of injecting the implant 12, the indicia 19 would be imparted to a viewable surface 21 of the implant 12. The implant would be positioned in an injection device which allows for positioning of the viewable surface 21 toward outside of the eye where it can be read as shown in FIGS. 3 and 4. The device 10 would be implanted through injection into a portion of the cornea or clear tissue 17 of the eye, outside the refractive zone, with its viewable surface 21 facing the exterior of the eye. Thereafter, the indicia 19 or the luminescent portion 11 would be readable through the clear eye tissue as noted above.

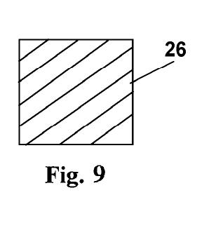

In yet another particularly preferred mode in FIG. 9, a luminescent material 26 that luminesces or glows under certain light wavelengths can be engaged to the device 10. This will allow the presence of the implant device 10 to be visually ascertained close up or a few feet away by simply shining the correct wavelength of light at the eye of the implanted, which will reveal the location of the device 10 by glowing. By providing implants 12 formed of a UV or IR fluorescent, or other wavelength fluorescent specific material which can only be read when illuminated at a certain frequency, such as in the UV range, the report by the implant 12 and the discerning of presence and/or reading of the implant 12 of the device 10 can be limited to medical professionals who employ scanning or viewing devices set to the particular wavelength. Such material is commonly available in plastic and polymer configurations with a glow additive such as with polyethylene, polypropylene, polyester, nylon, and other materials. Therefor, any passerby on the street could not view the implant 12 or discern its presence if formed of material which only luminesces when struck with light of a particular frequency which would be provided to the emergency personnel and users by the system operator providing the implants 12 and storing the information. Thus, privacy can be maintained.

However, in additional modes, the luminescent material 26 of the device 10 can include one or a combination of a luminescent material, glowing or fluorescence material, and a light reflective surface (such as metal or metallic flakes) or a prismatic shape reflecting light in colors, such that a visual reporting of the presence of the implant 12, by the light emanating from the body of the implant or attachments thereto, can be viewed with the naked eye. The material forming the implant, or at least a portion thereof or attached thereto, is thus a material which when struck by light, will emit a colored or glowing or other identifying reflection in a visual reporting of the presence of the implant which may be seen in daylight or darkness but will not be seen absent communication of light to the implant 12.

The device and method for storage and retrieval of patient medical records in the eye as disclosed and described herein, discloses steps in a process, arrangements of elements of particular construction, and configuration for illustrating preferred embodiments of structure and method of operation of the present invention. It is to be understood, however, that elements of different construction and configuration and different steps and process procedures and other arrangements thereof, other than those illustrated and described, may be employed for providing programming updates of interrelated electronic components with the spirit of this invention.

For example, in accordance with at least one other preferred mode, a particular method for retrieving patient information for emergency medical care is provided. In emergency medical situations, such as a response from an ambulance, the need for important prior medical records is of even greater importance since these types of situations may directly dictate the life or death of a patient with little time to spare, and correct actions must be taken immediately. Patient conditions, illnesses, allergies, etc. is information that emergency and treating personnel frequently lack but would desire.

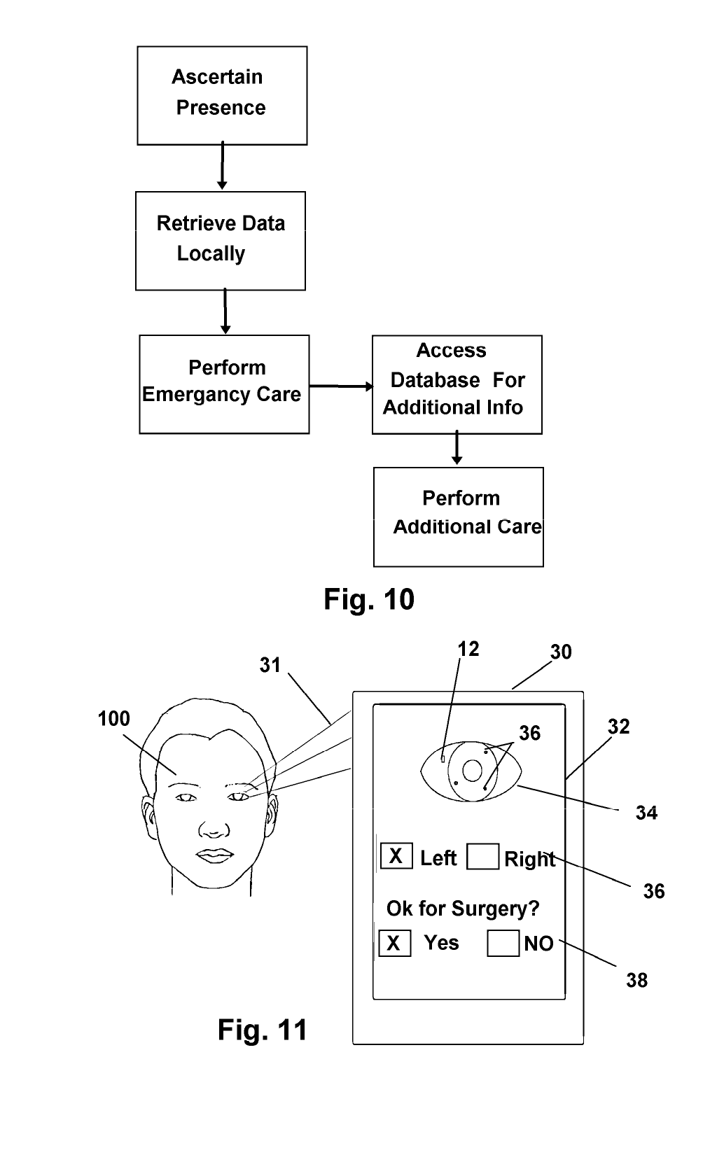

In this method shown in FIG. 10, the user, such as an emergency medical technician (EMT), will identify the presence of the device 10 through methods discussed previously such as shining a flashlight or a device emitting a particular light wavelength into the eye and visually discern a visual cue of the presence of the implant. As a service or method, both the implants 12 and the lights which react with them to provide a visual reporting of presence, are provided by the company hosting the website for the records or maintaining the database of records for mating to individuals according to implant 12 derived information.

The implant 12 of the method and device 10 herein preferably includes readable indicia 19, to allow the medical or other professional to read the medical and other information directly. Alternatively or in combination with the implant 12 with indicia information, an RFID 23 which can be positioned with electronic memory can provide the EMT with permanent patient information which can be retrieved locally, from the patient's person, on site without the need to access a network, phone system, server or database of patient records.

The ability to directly read patient information, directly from the implant 12, and optionally from a secondary source such as an RFID 23, is of great advantage due to the local retrieval and on site viewing of patient records. The indicia on the implant 12 readable by the third party through the clear layer of the eye may also include means to link to the a remote database provider and download more detailed patient information for further care, such as via providing correct patient ID numbers and/or correct identification codes which can be communicated to a remote inquirer such as the incoming medical facility or other professional who has access to such a patient history database.

Additionally provided by the service provider will be an attachment to a smartphone or camera to allow users to employ a microscopic or magnifying lens to read stored information illustrated by the indicia on the implant 12. If a magnifying lens is supplied, it could be engaged to the lens of the smartphone or computer camera employing a fiberoptic or optical communication.

Further, as noted previously, it is an additional intended object of the invention to provide a means for positive eye identification for patients undergoing ophthalmic surgery. Shown in FIG. 11, a patient's eyes 100 can be scanned 31, or an image captured, through the employment of a handheld electronic computing device 30 with a display, employing software adapted at the task. A viewing screen 32 on the electronic device 30 may display a captured or scanned 31 eye image 34 which may indicate the presence of the implant 12. Through additional RF or other communication means with the implant 12 as outlined in the previous modes above, the electronic device 30 can retrieve patient information regarding the implant location 36, and if the eye has been confirmed for surgery 38. Thus, surgical teams are afforded with a means for positive identification of a patient's eyes prior to surgery, which can substantially reduce the occurrence of surgical mistakes.

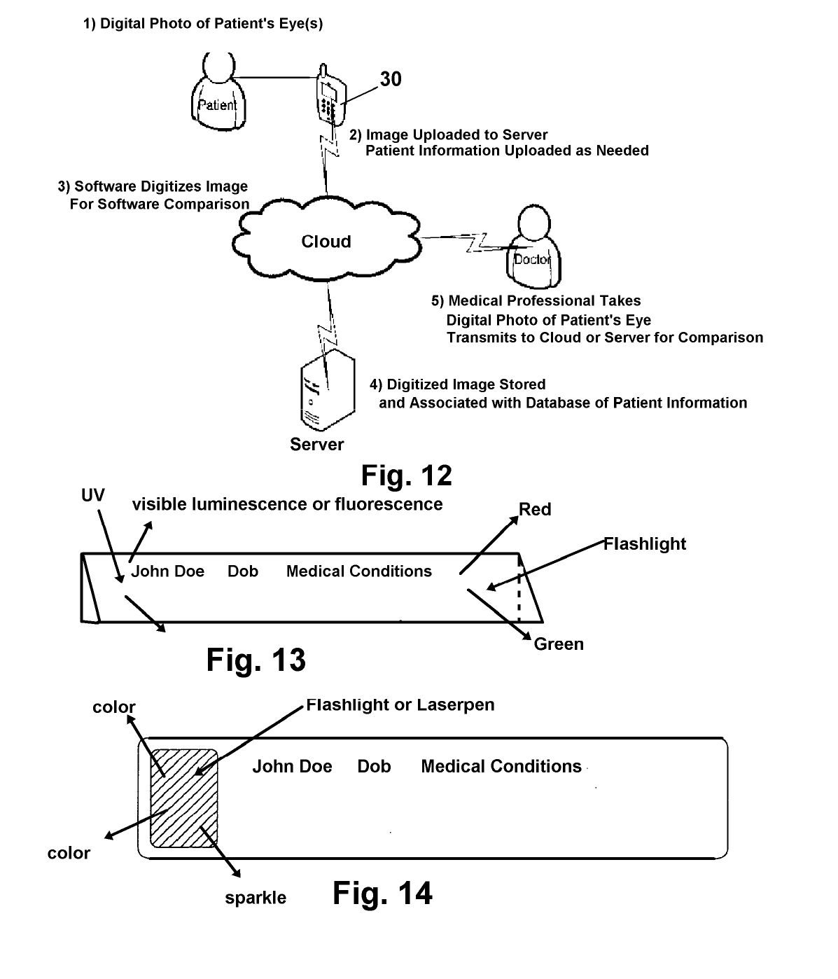

A preferred communication network diagram is shown in FIG. 12. As shown, the information captured by the device 30 can be communicated over a wireless network to a remote server, such as cloud network, which allows for easy retrieval by a doctor or surgical team as needed. However, it is noted that the invention can employ other communication means, and should therefor not be considered limited to that shown in the figure.

As a secondary means for positive identification of the patients eye 100 an iris or retina mapping 36 can be performed to further confirm the identification. Similar to fingerprints, the iris and retina have distinguishing markings and configurations which can be determined by software adapted at the task. By storing this mapping 36, and linking it to patient information, a means for a ‘digital fingerprint’ of the eye can be provided for additional identification purposes.

In FIG. 13 there is shown a mode of the implant 12 formed of a material in a shape for prismatic reflections or of material which fluoresces or is luminescent under certain light spectrums directed at it providing a lighted visual cue of presence which works well in daylight but especially well at night to visually report its presence.

FIG. 14 shows a mode of an implant 12 having a body 27 with readable indicia on a display surface. The body 27 which may be formed of material which luminesces or fluoresces or with a reflective element which reflects colored or sparkling light to visually report the presence of the implant.

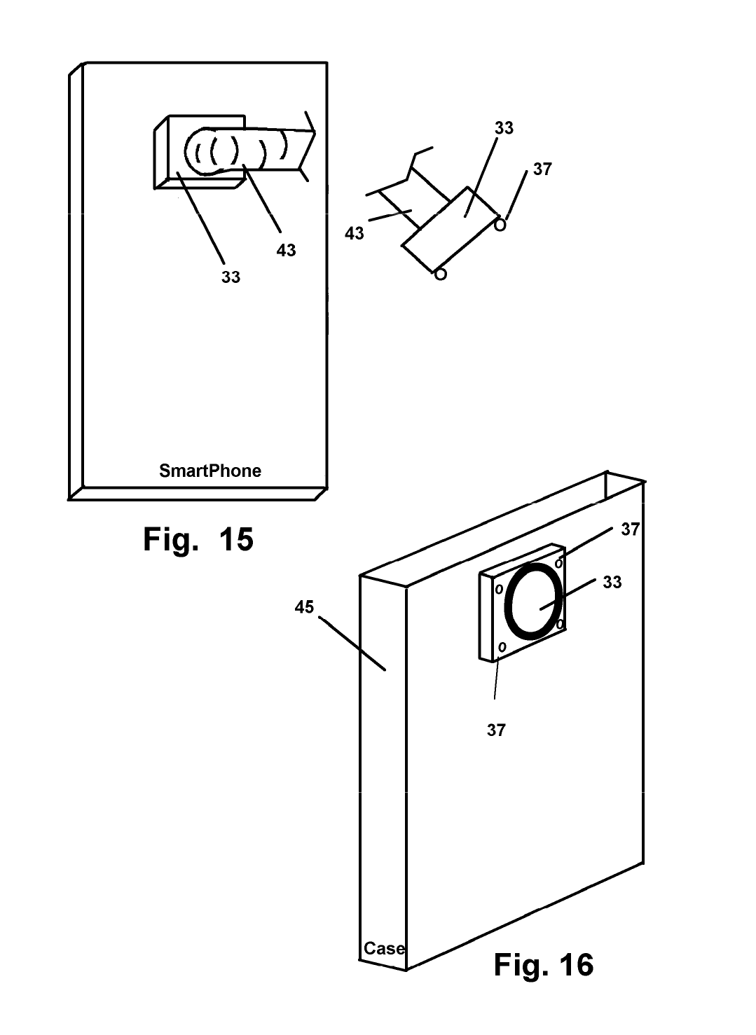

FIG. 15 depicts a magnifying lens attachment 33 and illumination components 37 which may be mounted to work with in conjunction with the camera lens of a smartphone 41 or portable computer or camera, and optional fiber optic lens 43 for extension of the magnifying lens.

FIG. 16 shows a mode of the device which includes a smartphone cover 45 or portable computer case or cover 45, which has a magnification lens 33 and illumination component 37 positioned to register with and configured to work with the OEM camera lens of a smartphone 41 or the like inserted within the cavity 47 to magnify the written indicia and display it on the display screen. Illumination components 37 for strobe, UV, IR, light or other noted light projection spectrums are also provided.

As such, while the present invention has been described herein with reference to particular embodiments thereof, a latitude of modifications, various changes and substitutions are intended in the foregoing disclosure, and it will be appreciated that in some instance some features of the invention could be employed without a corresponding use of other features without departing from the scope of the invention as set forth in the following claims. All such changes, alternations and modifications as would occur to those skilled in the art are considered to be within the scope of this invention as broadly defined in the appended claims.

What is claimed is:

1. A method of storage of directly readable information concerning a patient on an implant configured for implantation into a viewable position in the eye, of said patient, comprising:
imparting indicia concerning a patient to an implant upon a viewable surface thereof, said implant having an exterior surface defining a dimension; and
implanting said implant bearing said indicia to a mounting position, said mounting position locating said implant within tissue of the eye in a position enabling a reading of said indicia from its location upon said viewable surface through a clear tissue portion of the eye, whereby said patient information while in said mounted position may be read by a third party from a position exterior to the eye, to provide said patient information thereto without any communication to said third party by said patient.

2. The method of storage of directly readable information concerning a patient of claim 1 wherein said implanting is accomplished by the steps of:
forming a cavity in said clear tissue the eye which is adapted to engage said dimension of said implant and maintain it in a fixed position; and
implanting said implant in said cavity with said viewable surface facing away from the retina and toward said position exterior to the eye and viewable through said clear tissue from said position exterior to the eye, subsequent to implantation.

3. The method of storage of directly readable information concerning a patient of claim 1 additionally comprising:
forming said implant at least partially from material which upon communication of light thereto, illuminates, to provide a visual report of a presence of a said implant.

4. The method of storage of directly readable information concerning a patient of claim 3 additionally comprising:
providing said patient and/or said third parties with illumination components configured for a communication of said light to said implant through tissue of said eye, from said position exterior to said eye;
allowing said third party ascertain a presence of said implant upon a viewing said visual report during said communication of light to said implant.

5. The method of storage of directly readable information concerning a patient of claim 2 additionally comprising:
forming said implant at least partially from material which upon a communication of light thereto, illuminates, to provide a visual report of a presence of a said implant.

6. The method of storage of directly readable information concerning a patient of claim 5 additionally comprising:
providing said patient and/or said third parties with illumination components configured for a communication of said light to said implant through tissue of said eye, from said position exterior to said eye;
allowing said third party ascertain a presence of said implant upon a viewing said visual report during said communication of light to said implant

7. The method of storage of directly readable information concerning a patient of claim 2 wherein forming a cavity in said clear tissue of the eye is accomplished by the steps of:
employing one of a microsphereal injection of the implant or surgical incision and insertion therein of the implant, within cornea or other clear tissue of the eye.

8. The method of storage of directly readable information concerning a patient of claim 5 wherein forming a cavity in said clear tissue of the eye is accomplished by the steps of:
employing one of a microsphereal injection of the implant or surgical incision and insertion therein of the implant, within cornea or other clear tissue of the eye.

9. The method of storage of directly readable information concerning a patient of claim 6 wherein forming a cavity in said clear tissue of the eye is accomplished by the steps of:
employing one of a microsphereal injection of the implant or surgical incision and insertion therein of the implant, within cornea or other clear tissue of the eye.

10. The method of storage of directly readable information concerning a patient of claim 4 further comprising:
providing said third parties said illumination components upon or adjacent a magnification lens configured for optically correct engagement over a camera lens of a camera of a smartphone or hand held computing device; and
allow said third party to employ said illumination components to communicate light to an eye and subsequent to determine a presence of a said implant, allowing said third party to capture video depiction of said indicia with said camera lens as communicated through said magnification lens and to display such indicia on a display screen operatively connected with said camera.

11. The method of storage of directly readable information concerning a patient of claim 6 further comprising:
providing said third parties said illumination components upon or adjacent a magnification lens configured for optically correct engagement over a camera lens of a camera of a smartphone or hand held computing device; and
allow said third party to employ said illumination components to communicate light to an eye and subsequent to determine a presence of a said implant, allowing said third party to capture video depiction of said indicia with said camera lens as communicated through said magnification lens and to display such indicia on a display screen operatively connected with said camera.

12. The method of storage of directly readable information concerning a patient of claim 10 further comprising:
providing said illumination components which emit light in the UV or IR spectrum; and
instructing said third parties to look for luminescence instructing or fluorescence of all or part of said implant upon communication of light thereto.

13. The method of storage of directly readable information concerning a patient of claim 10 further comprising:
providing said illumination components which emit light in the UV or IR spectrum; and
instructing said third parties to look for luminescence instructing or fluorescence of all or part of said implant upon communication of light thereto.

14. The method of storage of directly readable information concerning a patient of claim 10 further comprising:
providing said third parties said illumination components upon or adjacent a magnification lens, seated upon an exterior surface of a casing having said magnification lens configured for optically correct engagement over a camera lens of a camera of a smartphone or hand held computing device; and
allow said third party to employ said illumination components to communicate light to an eye and subsequent to determine a presence of a said implant, allowing said third party to capture video depiction of said indicia with said camera lens as communicated through said magnification lens and to display such indicia on a display screen operatively connected with said camera.

15. The method of storage of directly readable information concerning a patient of claim 11 further comprising:
providing said third parties said illumination components upon or adjacent a magnification lens, seated upon an exterior surface of a casing having said magnification lens configured for optically correct engagement over a camera lens of a camera of a smartphone or hand held computing device; and
allow said third party to employ said illumination components to communicate light to an eye and subsequent to determine a presence of a said implant, allowing said third party to capture video depiction of said indicia with said camera lens as communicated through said magnification lens and to display such indicia on a display screen operatively connected with said camera.

16. The method of storage of directly readable information concerning a patient of claim 14 further comprising:
providing a fiberoptic cable communicating between said magnification lens and said camera lens.

17. The method of storage of directly readable information concerning a patient of claim 15 further comprising:
providing a fiberoptic cable communicating between said magnification lens and said camera lens.

18. The method of storage of directly readable information concerning a patient of claim 14 further comprising:
maintaining a database of digitized eye photographs of said patients and supplemental patient information;
having said third party employ said camera to capture an image of one or both eyes of the patient prior to an eye surgery and communicate such captured images to a server;
employing any patient identifying indicia to identify said patient and match them to any stored medical records;
employing software adapted to compare each said captured image in a comparison with known images in said database and to notify third parties if the eye captured in the image is the proper eye for surgery prior to surgery.

19. The method of storage of directly readable information concerning a patient of claim 15 further comprising:
maintaining a database of digitized eye photographs of said patients;
having said third party employ said camera to capture an image of one or both eyes of the patient prior to an eye surgery and communicate such captured images to a server;
employing software adapted to compare each said captured image in a comparison with known images in said database and to notify third parties if the eye captured in the image is the proper eye for surgery prior to surgery.

20. An apparatus for storage information in the eye comprising:
an implant having an exterior surface defining a dimension of said implant;
indicia concerning patient identity or health imparted to a viewable surface of said implant;
said implant implantable to a mounting position within a clear portion of eye tissue with said viewable surface facing an exterior of the eye; and
said viewing surface with said implant in said mounting position being readable using a magnifier from a position adjacent to the exterior surface of the eye, said inicia providing patient medical and personal information.
